(19) United States
(12) Patent Application Publication
(10) Pub. No.: US 2004/0043048 A1
(43) Pub. Date: Mar. 4, 2004

(54) Cosmetic containing anti-bacterial agent and process for production thereof

(75) Inventors: Makio Nomura, Nagoya-shi (JP); Reiko Nojima, Osaka (JP); Togo Murata, Sakura-shi (JP)

Correspondence Address: Law Offices of Townsend & Banta, Suite 900, 601 Pennsylvania Avenue, N.W., Washington, DC, 20004, US

(73) Assignees: Ishizuka Garasu Kabushiki Kaisha; SHIN-EI CHEMICAL CO., LTD.; Cosmo Trends Corporation

(21) Appl. No.: 10/653,897

(22) Filed: Sep. 4, 2003

(30) Foreign Application Priority Data
Sep. 4, 2002 (JP) 2002-259378

(51) Int. Cl. A61K 33/38; A61K 7/00

(52) U.S. Cl. 424/401; 424/618

(57) ABSTRACT

Disclosed herein are (1) cosmetic made with silver type anti-microbial water soluble glass in oily cosmetic composition of the cosmetic, and (2) a process for producing the cosmetic that is highly effective against bacteria and fungi, and that is safer to the skin. The cosmetic is characterized by letting silver type anti-microbial water soluble glass in oily cosmetic composition release slowly from oil phase of the cosmetic to the water phase of the cosmetic only when anti-bacterial agent contact with water in the cosmetic.

### RELATED APPLICATION

This application claims the priority of Japanese Patent Application No. 2002-259378 filed on Sep. 4, 2002.

### BACKGROUND OF THE INVENTION

1. Field of the Invention

The present invention relates to cosmetics that include silver-type anti-bacterial agents having an excellent long-term stability and being safer in use to inhibit multiplication of microorganisms, and a process for production thereof.

2. Description of Related Art

Conventionally, such cosmetics as toilet water, milky emulsion, cream and emulsion-type foundation rich in water and nutrient tend to allow microorganisms such as bacteria and fungi to grow, and are vulnerable to putrefaction by these microorganisms. In addition, during production, distribution and consumers' consumption of such cosmetics, there is a possibility of contingent contamination of cosmetics with microorganisms. When microorganisms are mixed in cosmetics and grow therein afterwards, there are possibilities of deterioration of cosmetic quality, skin disorder caused by change in ingredients, infection by disease germs, opportunistic infection by non-disease germs and hazardous actions by microorganisms' component or metabolic product.

There are also possibilities of the same hazardous actions described above even in cosmetics such as oily foundation, cake-type foundation and lipsticks that do not accompany water in them because of adherence of moisture in the air onto these cosmetics and accidental interfusion of water in any form.

There are further possibilities of the same hazardous actions in no microorganisms' contaminated cosmetics if bacteria and fungi in the moisture occurring in human sweat and in water used for application of these cosmetics grow and proliferate on the skin after cosmetics are applied.

Hence, it becomes necessary to inhibit growth of microorganisms that accidentally interfuse into cosmetics, to sterilize them and to prevent their growth and proliferation in cosmetics through the whole period of production and distribution of cosmetics until consumers' complete use of these cosmetics.

At present it is common to use organic preservatives such as parabens and phenoxyethanol in cosmetics. However, if the organic anti-bacterial agents are mixed in quantity enough to bring out sufficient effect, they often cause short-time stimulation, sensitization on skin, and problems of giving uncomfortable feelings when using cosmetics.

Different from the organic anti-bacterial agents described above, inorganic anti-bacterial agents represented by metallic anti-bacterial agents are safer and more excellent in effect. Especially among them, what have been popular are silver-type anti-bacterial agents that are effective in less quantity than organic ones and that have advantages to reduce short-time stimulation and sensitization to the skin. As silver-type anti-bacterial agents, there have been preparations comprising inorganic fillers such as zeolite or calcium phosphate with silver ions or metal silver. Other silver type anti-bacterial agents have been known in Japanese Patent Application Laid-open No. 2001-247425 disclosing silver-chloro complex salt and in Japanese Patent Application Laid-open No. H04-270208 disclosing the use of anti-microbial water soluble glass in cosmetics.

However, above-mentioned inorganic anti-bacterial agents have characteristics that they release a lot of silver ion as anti-microbial component into the water phase of cosmetics. This characteristic causes silver component to elute into water phase during production of cosmetics, and anti-microbial effect cannot be maintained until use of cosmetics. Even if silver component remains until use of cosmetics, the remaining silver component elutes soon after use of the cosmetics because the anti-bacterial agents have increasing chances to contact with water after use, leading to a possibility that anti-microbial effect does not last during use of cosmetics. Thus, it gives rise to adding more silver component than required actually in order to maintain effect during use, resulting in higher cost of production.

Among many kinds of cosmetics, some cosmetics are weak against fungus and need more anti-bacterial agents than other kind of cosmetics. Those cosmetics include emulsion obtained by emulsifying water and oil with surface active agents, especially water-in-oil emulsion obtained by emulsifying water into oil. However, it becomes costly to add more anti-bacterial agents into emulsion than other cosmetics to strengthen anti-microbial effect. In addition, even safer silver type anti-bacterial agents cause skin irritation and sensitization when used excessively.

### SUMMARY OF THE INVENTION

The present invention was completed to solve the above-mentioned problem. It is an object of the invention to provide cosmetics having a bit of silver type anti-microbial water soluble glass in oily cosmetic composition of the cosmetics whereby maintaining a broad anti-microbial spectrum over a long period of time from production to use of the cosmetics.

To achieve the above object, the cosmetics according to this invention comprise oily cosmetic composition in which silver type anti-bacterial agent is dispersed in oil phase.

In the case of above-mentioned oily cosmetic composition that is produced by dispersing silver type anti-bacterial agent in oil phase, the agent is surrounded by the oil phase, whereby the agent does not release the anti-microbial component in oil phase. Thus, the anti-bacterial agent in oil phase has less probability to contact with water where bacteria and fungi grow. Meanwhile, the anti-bacterial agent releases anti-microbial component only when it contacts with water. Accordingly, the anti-microbial component is set to elute bit by bit into water phase over a long period of time from the point of production to use without releasing a large quantity of the component into water phase for a short period of time. This phenomenon is referred to as “extended release”.

It is another object of the present invention to provide a process for producing the cosmetics that are highly effective against bacteria and fungi and that safer to the skin.

According to the present invention, the silver type anti-bacterial agents are the materials that contain silver as anti-microbial metal, silver ion or the both. Examples of the materials are chemical compound such as silver nitrate, silver chloride, silver sulfate, silver lactate, silver acetate, silver ions such as silver ammonium complex, silver chloro complex, silver thiosulfato complex, and materials that comprise silver, silver ion, chemical compound containing silver in soluble glass, glaze, zirconium phosphate, calcium phosphate, aluminum phosphate, apatite, zeolite and the like. Especially, the materials that comprise silver or silver ion in soluble glass, glaze, zirconium phosphate, calcium phosphate, aluminum phosphate, apatite, zeolite are desirable since they are easy to handle and control elution speed compared with silver ion or chemical compounds containing silver.

Further desirable of the materials that carry above mentioned silver or silver ion is the material that comprises soluble glass, namely silver type anti-microbial water soluble glass is the most desirable in terms of balancing anti-microbial activity and usability over a long period.

The silver type anti-microbial water soluble glass is particulate with the average particle size ranging from 0.01 μm to 20 μm. With the particle size under 0.01 μm, the elution speed of anti-microbial component is too fast. With the size over 20 μm the speed is too slow and it may impart abrasion to the skin when applied on the skin. Further, it is desirable that the average particle size of the glass ranges from 0.1 μm to 12 μm. It is particularly desirable to be between 0.1 μm and 10 μm.

Silver content in the water soluble glass of the invention is not limited specifically since it can be adjusted to the purpose and use accordingly, but it is desirable to set the silver content in the soluble glass between 0.01 wt % and 10 wt %. While the antimicrobial activity becomes weak with less than 0.01 wt %, it becomes disadvantageous in cost to incorporate more than 10 wt %.

Oil phase materials of the invention are not specifically limited, but it is desirable to use oily material approved for use in cosmetics. Examples of the oily materials are solid hydrocarbon such as paraffin, liquid hydrocarbon such as liquid paraffin and squalane, pasty hydrocarbon such as Vaseline, higher fatty acid such as stearic acid, higher alcohol such as lanolin alcohol, cetostearyl alcohol, vegetable oil and animal oil such as shea butter, synthetic ester oil such as isopropyl myristate, wax such as liquid lanolin, bees wax, carnauba wax, unsaturated hydrocarbon such as polybutene, fluorinated oil such as silicone oil. These oily materials can be used singly or as the mixture of more than two materials. What kinds of oily materials are used is decided by the form of cosmetics either in solid, liquid or semi-solid or usability and feeling in use.

### BRIEF DESCRIPTION OF THE DRAWINGS

FIG. 1 is an enlarged schematic view of a model of oily foundation;

FIG. 2 is an enlarged schematic view of a model of water-in-oil emulsion foundation;

FIG. 3 is an enlarged schematic view of a model of oil-in-water emulsion foundation: and

FIG. 4 is an enlarged schematic view of a model of cake type foundation.

### DESCRIPTION OF THE PREFERRED EMBODIMENT

The cosmetics of the invention is made with the above mentioned oily cosmetic composition and described in details below.

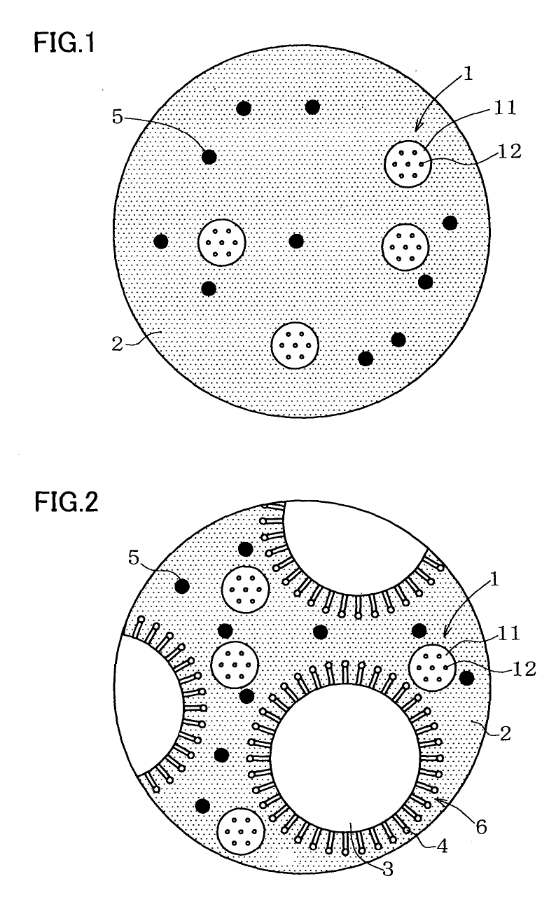

The first form of the cosmetics is the cosmetic in which powdery pigments are dispersed in the oily cosmetic composition. Examples of such cosmetics are oily foundation and lipsticks, which are made by mixing several kinds of oily materials. It is possible to produce cosmetics of varied hardness by changing kinds of oily materials and mixing ratio. Examples of the oily foundation are pasty oil foundation and solid oily stick foundation. The oily foundation is made by heating the oily cosmetic composition to its melting temperature or around (for example, to 70-80° C. depending the oily materials), blending the powdery pigments into the melted oily cosmetic composition, and then pouring the mixture into a shallow case into paste, or pouring the mixture into mold or case that can be moved up and down into solid, for example, a stick. Silver type anti-microbial water soluble glass blended in the oily foundation exerts anti-microbial effect especially during use of the cosmetics since the cosmetics do not contain moisture in them. The anti-bacterial agent exerts anti-microbial effect during and after use of the cosmetics with a puff wet with water or sweat, and so does on the product to which sweat or moisture in the air adhered. In these cases, water adheres to the surface of the cosmetic, providing the space for microorganisms to grow and proliferate. But silver component gradually elutes toward water on the surface of the oil phase and maintain anti-microbial effect over a long period of time. The model of oily foundation is illustrated in the FIG. 1.

The second form of the cosmetics is cosmetics that contain emulsion by emulsifying the oily cosmetic composition and aqueous cosmetic composition with surface active agents. Examples of the cosmetics are emulsion type foundation, toilet water, milky emulsion and creams.

The kinds of emulsion are largely classified into two categories that are water-in-oil emulsion where aqueous cosmetic composition is dispersed in oily cosmetic composition and oil-in-water emulsion where oily cosmetic composition is dispersed in aqueous cosmetic composition. The two kinds of emulsion are defined by the hydrophile-lipophile balance (HLB) of the surface active agents, oil content, water content, the order and speed of adding the both compositions. The emulsion is made by adding properly selected surface active agent into both or either of water phase and oil phase and then adding oily composition into aqueous composition or aqueous composition into oily composition while stirring. Usually, both cosmetic compositions are heated to its melting temperature or around, for example, to 65-85° C., depending on the kind of oily ingredients. After adding the composition, stirring is kept until the mixture is cooled down to room temperature. This is how the emulsion is made. When the emulsion is in the form of water-in-oil emulsion, there are a lot of micelle of aqueous composition surrounded by the surface active agent in the oil phase. In the oil-in-water emulsion the phases are reversed. In the cosmetics of the invention, silver type anti-bacterial agent is dispersed in the oil phase of the cosmetics. The particle diameter of the micelle or emulsion is desirable to be between 0.3 μm and 20 μm. It is not common to produce micelle whose particle diameter is under 0.3 μm since it needs higher mechanical sheer, while micelle over 20 μm is not suitable since each micelle may associate to larger micelles.

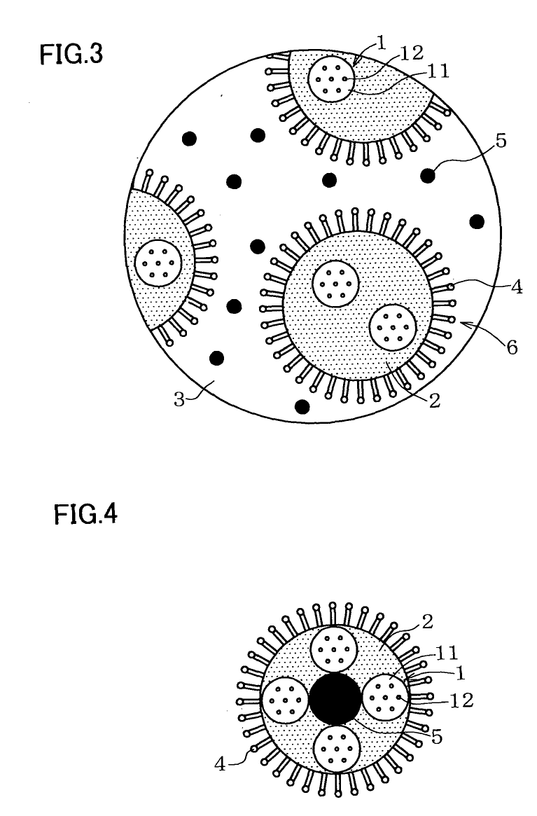

In the cosmetics of the invention, powdery pigments are dispersed in the above-mentioned emulsion, and there are processes for dispersing the powdery pigments described below. In case of emulsion type foundation, there are following methods of producing the emulsion. The first one is to produce emulsion type foundation by blending powdery pigments either in the oily cosmetic composition or aqueous cosmetic composition, then emulsifying the both composition with surface active agents. More practically, powdery pigments are dispersed in either oil phase or aqueous phase before emulsifying in such method as powdery pigments with surface active agent are dispersed in the base phase (oil phase if it is water-in-oil emulsion), then the other phase (aqueous phase if it is water-in-oil emulsion) is mixed into the other phase while stirring. The second method to produce emulsion type foundation is that after emulsion is produced from the oily cosmetic composition and aqueous cosmetic composition with surface active agents, the powdery pigments are dispersed into the emulsion. This is the method to disperse and blend the powdery pigments after emulsion is produced. This latter method is applicable to liquid emulsion since it is hard to add the pigments into emulsion that does not have fluidity. FIG. 2 illustrates a model of a water-in-oil emulsion, FIG. 3 an oil-in-water emulsion.

Emulsion type foundation is produced by mixing one or more oily ingredients, aqueous ingredients, surface active agents and powdery pigments, and it is possible to produce emulsion of various degree of viscosity by changing the mixing ratio and kind of oily ingredients, and changing the content of aqueous ingredients. As emulsion type foundations are milky emulsion foundation where the above-mentioned method of dispersing powdery pigments into emulsion is applicable, and creamy emulsion foundation where the method is hard to apply because of no fluidity of the emulsion.

Aqueous materials to comprise aqueous cosmetic composition used in emulsion type cosmetics of the invention are water soluble materials used in various cosmetics. Examples are purified water, lower alcohol such as ethanol, and polyalcohol such as glycerin, sorbitol and propylene glycol that are called moisturizing materials except for purified water. One or more selected from the above are used. Surface active agents that are adopted in their HLB can be exemplified with nonionic surface active agent such as decaglyceryl pentaoleate, decaglyceryl pentaisostearate, decaglyceryl diisostearate, sorbitan fatty acid ester, polyoxyethylene alkyl ether, cationic surface active agent such as quaternary ammonium salt, and silicon surface active agents, all of which are publicly known. These surface active agents can be used singly or as the mixture of two or more agents.

In the emulsion type foundation of the invention, anti-microbial effect is exerted when used and especially during over a long period of storage when microorganisms tend to grow and proliferate in the water phase of the cosmetics.

Another form of cosmetics in which powdery pigments are dispersed is a cake type foundation where powdery pigments are coated with the oily cosmetic composition and surface active agents of the invention. The cake type foundation is emulsified when it is used with water for application onto the skin. Some of cake type foundation can be emulsified by combination of base cream. Other cake type foundation called Two Way cake can be used with or without water for application.

Cake type foundation is made by coating powdery pigments with the oily cosmetic composition and surface active agents, then arranging the particle size of the pigments, and pressing the mixture into a cake. Particle size of the pigments is uniformly arranged by means of sieve between 0.1 μm and 10 μm. It becomes hard to match color with particle size under 0.1 μm while particle over 10 μm impart coarse feel when applied. It is preferred that particle size ranges between 0.1 and 3.0 μm, particularly preferred between 0.1 and 1.0 μm. In the cake type foundation of the invention, anti-microbial effect is exerted during its storage and especially on its use when water is applied. FIG. 4 illustrates the model of a cake type foundation.

Those useable as the powdery pigments of the invention include, for example, extender pigments (such as talc, mica, sericite, kaolin, silica, polystyrene powder, polymethyl methacrylate powder, nylon powder), inorganic color pigments(such as titanium dioxide, zinc oxide, iron oxide, cobalt oxide, chromium oxide, ultramarine blue, titanated mica, prussian blue), organic tar color pigments, inorganic or organic dyes. They may be used singly or as the mixture of two or more of them. These pigments are crushed in advance in pulverizers before dispersing into cosmetics. Pigments that are not lipophilic are treated to impart hydrophobicity before dispersing into oily ingredients. Hydophobicity can be imparted by adhering oily ingredients to the surface of the pigments by heating or by adsorbing surface active agents to their surface. These treatments can be exemplified by silicon treatment, metal soap treatment, paraffin treatment, chitosan treatment, higher fatty acid treatment.

Powdery pigments of the invention are preferred to be in fine particle size in order to achieve stable dispersion in the cosmetics. The particle size is preferably between 0.1 μm and 20 μm. It becomes hard to match color with particle size under 0.1 μm while particle over 20 μm impart coarse feel when applied. It is preferred that particle size ranges between 0.1 and 3 μm, particularly preferred between 0.1 and 1 μm. Especially the particle size is preferred to be smaller than the size of micelle of the emulsion type foundation. Publicly known methods such as hammer mill, beads mill, ball mill, jet mill and pin mill can be adopted to make particle finer.

The cosmetics of the invention embrace, if necessary, additives used commonly in cosmetics such as ultraviolet ray absorber, perfume, thickner, cleaner, foaming agent, antiformer.

The present invention is applicable to various cosmetics in the forms of liquid, solid and semi-solid that contain oily cosmetic composition, and more particularly oily foundation, emulsion type foundation, cake type foundation, hand cream, face cream, hair cream, body cream, sun care cream, mascara, eyeliner, cheek color, eye shadow, lipstick in stick and liquid.

The invention will be described in more details with reference to the following examples, in which silver borosilicate, silver type anti-microbial water-soluble glass in the description is all processed to about 10 μm in average particle size.

### EXAMPLE 1

A creamy, water-in-oil emulsion cosmetic was prepared with ingredients listed in Table 1 including 0.03 wt % of silver borosilicate, silver type anti-microbial water-soluble glass. To be more precise, a mixture A was prepared from oily ingredients 1-6 in Table 1, surface active agents 7-9 and silver borosilicate, silver type anti-microbial water-soluble glass 13. Then, a mixture B was prepared from purified water 10 listed in Table 1 and a mixture of humectants 11-12. After both mixtures were heated to 70° C., the mixture B was poured into the mixture A while stirring it to emulsify the whole and the composite of the two mixtures was cooled down.

### EXAMPLE 2

A cosmetic was prepared with ingredients listed in Table 1 including 0.05 wt % of silver borosilicate, silver type anti-microbial water-soluble glass. The compounding ratio of basic ingredients and method of preparation were in the same manner as in the example 1.

### EXAMPLE 3

A cosmetic was prepared with ingredients listed in Table 1 including 0.1 wt % of silver borosilicate, silver type anti-microbial water-soluble glass. The compounding ratio of basic ingredients and method of preparation were in the same manner as in the example 1.

### COMPARATIVE EXAMPLE 1

A cosmetic was prepared with ingredients listed in Table 1 including 0.2 wt % of methyl paraben and 0.05 wt % of propyl paraben, both organic anti-bacterial agents. The compounding ratio of basic ingredients and method of preparation were in the same manner as in the example 1.

### COMPARATIVE EXAMPLE 2

A cosmetic was prepared with ingredients listed in Table 1 including 0.3 wt % of methyl paraben and 0.05 wt % of propyl paraben and 0.05 wt % of butyl paraben, all the three organic anti-bacterial agents. The compounding ratio of basic ingredients and method of preparation are in the same manner as in the example 1.

### COMPARATIVE EXAMPLE 3

A cosmetic was prepared with ingredients listed in Table 1 including 0.3 wt % of methyl paraben and 0.1 wt % of propyl paraben and 0.1 wt % of butyl paraben, all the three organic anti-bacterial agents. The compounding ratio of basic ingredients and method of preparation are in the same manner as in the example 1.

### COMPARATIVE EXAMPLE 4

A cosmetic was prepared with ingredients listed in Table 1 including 0.8 wt % of phenoxy ethanol, organic anti-bacterial agents. The compounding ratio of basic ingredients and method of preparation are in the same manner as in the example 1.

TABLE 1
Formulation of creamy, water-in-oil emulsion foundation
unit = wt %

| | | | examples | | | comparative examples | | | |
|---|---|---|---|---|---|---|---|---|---|
| | ingredients | function | 1 | 2 | 3 | 1 | 2 | 3 | 4 |
| 1 | Octyl dodecyl isostealate | oil | 25 | same as | same as | same as | same as | same as | same as |
| 2 | Squalane | oil | 15 | example | example | example | example | example | example |
| 3 | Bee's wax | oil | 8 | 1 | 1 | 1 | 1 | 1 | 1 |
| 4 | Cholesteryl hydroxystearate | oil | 0.2 | | | | | | |
| 5 | Octyl dodecyl hydroxystearate | oil | 0 | | | | | | |
| 6 | shea butter | oil | 5 | | | | | | |
| 7 | Decaglyceryl pentaoleate | surfactant | 3 | | | | | | |
| 8 | Decaglyceryl pentaisostearate | surfactant | 0 | | | | | | |
| 9 | Decaglyceryl diisostearate | surfactant | 1 | | | | | | |
| 10 | Purified water | Base | to 100 | | | | | | |
| 11 | Glycerine | humectant | 3 | | | | | | |
| 12 | Solbitol | humectant | 0 | | | | | | |
| 13 | Silver borosilicate | anti-microbial active | 0.03 | 0.05 | 0.1 | — | — | — | — |
| 14 | Methy paraben | anti-microbial active | — | — | — | 0.2 | 0.3 | 0.3 | 0 |
| 15 | Propyl paraben | anti-microbial active | — | — | — | 0.05 | 0.05 | 0.1 | 0 |
| 16 | Butyl paraben | anti-microbial active | — | — | — | 0 | 0.05 | 0.1 | 0 |
| 17 | Phenoxy ethanol | anti-microbial active | — | — | — | 0 | 0 | 0 | 0.8 |

Anti-microbial activities were evaluated with creamy, water-in-oil emulsion cosmetics obtained in the examples 1,2 and 3 and comparative examples 1,2,3 and 4 of the invention. The results are shown in Table 2.

The evaluation was carried out by the single inoculation method with following strains.

(1) Staphylococcus aureus IFO No.13276

(2) Eschrichia Coli IFO No. 3972

(3) Pseudomonas aeruginosa IFO No. 1594

(4) Aspergillus terreus IFO No. 33024

First, each strain was cultivated in tryptic soy agar culture medium. Then, each cultivated strain was diluted into 4 ml of sterilized physiological saline to prepare inoculum. The prepared inoculum was inoculated to the level of (1) 1×10⁶, (2) 4×10^{6,} (3) 6×10⁶, (4) 5×10^{6,} (cells/g), stirred, kept at 25-27° C. for two weeks. In 1,2,7 and 14 days, each specimen was taken into 0.1 ml of tryptic soy agar culture medium to culture and measure the number of microorganisms.

TABLE 2

| | Day 1 | | | | Day 2 | | | | Day 7 | |
|---|---|---|---|---|---|---|---|---|---|---|
| kind of strain | 1 | 2 | 3 | 4 | 1 | 2 | 3 | 4 | 1 | 2 |
| example 1 | 0 | 3 × 10 | 8 × 10 | 0 | 0 | 0 | 0 | 0 | 0 | 0 |
| example 2 | 0 | 0 | 3 × 10 | 0 | 0 | 0 | 0 | 0 | 0 | 0 |
| example 3 | 0 | 0 | 0 | 0 | 0 | 0 | 0 | 0 | 0 | 0 |
| com. example 1 | * | 8 × 10⁵ | * | 2 × 10⁵ | * | 5 × 10⁴ | * | 5 × 10³ | * | 2 × 10⁵ |
| com. example 2 | 2 × 10⁵ | 5 × 10² | * | 6 × 10³ | 3 × 10⁴ | 2 × 10² | * | 5 × 10² | 2 × 10³ | 5 × 10 |
| com. example 3 | 3 × 10⁴ | 0 | * | 6 × 10³ | 2 × 10³ | 0 | * | 3 × 10² | 3 × 10² | 0 |
| com. example 4 | * | * | * | * | * | * | * | * | * | 6 × 10⁴ |

| | Day 7 | | Day 14 | | | |
|---|---|---|---|---|---|---|
| kind of strain | 3 | 4 | 1 | 2 | 3 | 4 |
| example 1 | 0 | 0 | 0 | 0 | 0 | 0 |
| example 2 | 0 | 0 | 0 | 0 | 0 | 0 |
| example 3 | 0 | 0 | 0 | 0 | 0 | 0 |
| com. example 1 | * | 3 × 10³ | * | 8 × 10² | * | 5 × 10² |
| com. example 2 | * | 4 × 10² | 3 × 10² | 5 × 10 | * | 6 × 10 |
| com. example 3 | 8 × 10⁴ | 5 × 10 | 8 × 10 | 0 | 2 × 10⁴ | 0 |
| com. example 4 | 7 × 10⁵ | * | * | 6 × 10³ | 5 × 10³ | * |

1 = S. aureus 1 × 10⁶
2 = E. Coli 4 × 10⁶
3 = P. aeruginosa 6 × 10⁶
4 = A. terreus 5 × 10⁶
unit = cells/g

Table 2 clearly shows that organic anti-bacterial agents used in the comparative examples 1-4 did not provide good anti-microbial efficacy since microorganisms in them did not disappear over a long period of time. On the other hand, the table shows that even a bit of silver borosilicate in the examples 1-3 of the creamy, water-in-oil emulsion cosmetics of the invention provided excellent anti-microbial efficacy since microorganisms in them virtually disappeared in one day with the status kept afterwards.

The same experiments as mentioned above were performed with cake-formed foundation in the invention.

### EXAMPLE 4

A two way cake foundation was prepared with ingredients listed in Table 3 including 0.02 wt % of silver borosilicate, silver type anti-microbial water soluble glass. Two way cake foundation is a kind of foundation that can be applied either with water or without water. To be more precise, ingredients 1-10 in Table 3 were uniformly mixed, then crushed in a pulverizer to get particle size into about 2 μm, and pressed into two way cake foundation. Pigments 5-10 in the table were silicone treated powder to have them hydrophobic.

### EXAMPLE 5

A two way cake foundation was prepared with ingredients listed in Table 3 including 0.05 wt % of silver borosilicate, silver type anti-microbial water soluble glass. The compounding ratio of basic ingredients and method of preparation are in the same manner as in the example 4.

### EXAMPLE 6

A two way cake foundation was prepared with ingredients listed in Table 3 including 0.1 wt % of silver borosilicate, silver type anti-microbial water soluble glass. The compounding ratio of basic ingredients and method of preparation are in the same manner as in the example 4.

### COMPARATIVE EXAMPLE 5

A two way cake foundation was prepared with ingredients listed in Table 3 including 0.3 wt % of methyl paraben. The compounding ratio of basic ingredients and method of preparation are in the same manner as in the example 4.

### COMPARATIVE EXAMPLE 6

A two way cake foundation was prepared with ingredients listed in Table 3 including 0.2 wt % of methyl paraben and 0.1 wt % of propyl paraben. The compounding ratio of basic ingredients and method of preparation are in the same manner as in the example 4.

### COMPARATIVE EXAMPLE 7

A two way cake foundation was prepared with ingredients listed in Table 3 including 0.2 wt % of methyl paraben and 0.1 wt % of propyl paraben and 0.1% of butyl paraben. The compounding ratio of basic ingredients and method of preparation are in the same manner as in the example 4.

### COMPARATIVE EXAMPLE 8

A two way cake foundation was prepared with ingredients listed in Table 3 including 0.1 wt % of methyl paraben and 0.6 wt % of phenoxy ethanol. The compounding ratio of basic ingredients and method of preparation are in the same manner as in the example 4.

TABLE 3
Two Way Cake Foundation

| | | unit = wt % | | | | | | |
|---|---|---|---|---|---|---|---|---|
| | | examples | | | comparative examples | | | |
| | Ingredients | 4 | 5 | 6 | 5 | 6 | 7 | 8 |
| 1 | Liquid parafin | 4.3 | same as | same as | same as | same as | same as | same as |
| 2 | Glyceryl trioctanoate | 1.8 | example | example | example | example | example | example |
| 3 | Wacelin | 1 | 1 | 1 | 1 | 1 | 1 | 1 |
| 4 | shea butter | 1 | | | | | | |
| 5 | Polyethylene wax | 1 | | | | | | |
| 6 | Silicone treated talc | to 100 | | | | | | |
| 7 | Silicone treated titanium oxide | 8 | | | | | | |
| 8 | Silicone treated mica | 52 | | | | | | |
| 9 | Silicone treated pigment | proper quantitiy | | | | | | |
| 10 | Silicone treated complex powder | 8 | | | | | | |
| 11 | Siliver borosilicate | 0.02 | 0.05 | 0.1 | — | — | — | — |
| 12 | Methyl paraben | — | | | 0.3 | 0.2 | 0.2 | 0.1 |
| 13 | Propyl paraben | | | | 0 | 0.1 | 0.1 | 0 |
| 14 | Butyl paraben | | | | 0 | 0 | 0.1 | 0 |
| 15 | Phenoxy ethanol | | | | 0 | 0 | 0 | 0.6 |

Anti-microbial activities were evaluated with Two Way Cake obtained in the examples 4, 5 and 6 of the invention and comparative examples 5,6,7 and 8 in the same manner as done for the case of water-in-oil emulsion foundation. The results are shown in Table 4.

TABLE 4

| | Day 1 | | | | Day 2 | | | | Day 7 | |
|---|---|---|---|---|---|---|---|---|---|---|
| kind of strain | 1 | 2 | 3 | 4 | 1 | 2 | 3 | 4 | 1 | 2 |
| example 4 | 0 | 2 × 10² | 5 × 10² | 0 | 0 | 3 × 10 | 2 × 10 | 0 | 0 | 0 |
| example 5 | 0 | 3 × 10 | 6 × 10 | 0 | 0 | 0 | 0 | 0 | 0 | 0 |
| example 6 | 0 | 0 | 0 | 0 | 0 | 0 | 0 | 0 | 0 | 0 |
| com. example 5 | * | 6 × 10⁶ | * | 5 × 10⁶ | * | 2 × 10⁵ | * | 3 × 10⁵ | * | 2 × 10⁵ |
| com. example 6 | * | 3 × 10³ | * | 6 × 10⁴ | 9 × 10⁵ | 3 × 10³ | * | 3 × 10³ | 5 × 10⁴ | 6 × 10² |
| com. example 7 | 4 × 10⁵ | 0 | 5 × 10⁴ | 3 × 10³ | 5 × 10³ | 0 | 6 × 10³ | 5 × 10² | 3 × 10² | 0 |
| com. example 8 | * | * | * | * | 5 × 10⁵ | 6 × 10⁵ | * | 3 × 10³ | * | 4× 10³ |

| | Day 7 | | Day 14 | | | |
|---|---|---|---|---|---|---|
| kind of strain | 3 | 4 | 1 | 2 | 3 | 4 |
| example 1 | 0 | 0 | 0 | 0 | 0 | 0 |
| example 2 | 0 | 0 | 0 | 0 | 0 | 0 |
| example 3 | 0 | 0 | 0 | 0 | 0 | 0 |
| com. example 1 | * | 8 × 10² | * | 2 × 10² | * | 8 × 10² |
| com. example 2 | * | 2 × 10² | 3 × 10² | 5 × 10 | * | 4 × 10 |
| com. example 3 | 8 × 10² | 5 × 10 | 3 × 10 | 0 | 2 × 10 | 0 |
| com. example 4 | 6 × 10⁵ | * | 4 × 10² | 3 × 10³ | 2 × 10³ | * |

1 = S. aureus 1 × 10⁶
2 = E. Coli 4 × 10⁶
3 = P. aeruginosa 6 × 10⁶
4 = A. terreus 5 × 10⁶
unit = cells/g

Table 4 clearly shows that organic anti-bacterial agents used in the comparative examples 5-8 did not provide good anti-microbial efficacy since microorganisms in them did not disappear over a long period of time. On the other hand, the table shows that even a bit of silver borosilicate in the examples 4-6 of two way cake foundation of the invention provided excellent anti-microbial efficacy since microorganisms in them virtually disappeared in one day with the status kept afterwards.

What is claimed is:

1. Cosmetic which comprises oily cosmetic composition wherein silver type anti-bacterial agent is dispersed.

2. Cosmetic according to claim 1 wherein the silver type anti-bacterial agent is silver type anti-microbial, water soluble glass.

3. Cosmetic according to claim 2 wherein the silver type anti-microbial, water soluble glass is particulate and having an average particle diameter of 0.01 μm to 20 μm.

4. Cosmetic according to claim 1 wherein powdery pigments are dispersed in the oily cosmetic composition.

5. Cosmetic according to claim 4 wherein the cosmetic is oily foundation.

6. Cosmetic according to claim 5 wherein the oily cosmetic foundation is in the form of ointment.

7. Cosmetic according to claim 5 wherein the oily foundation is in the form of solid stick.

8. Cosmetic according to claim 1 which comprises at least water-in-oil emulsion wherein aqueous cosmetic composition is dispersed in emulsion in the oily cosmetic composition.

9. Cosmetic according to claim 1 which comprises oil-in-water emulsion wherein the oily cosmetic composition is dispersed in emulsion in the aqueous cosmetic composition.

10. Cosmetic according to claim 8 wherein powdery pigments are dispersed in the emulsion.

11. Cosmetic according to claim 9 wherein powdery pigments are dispersed in the emulsion.

12. Cosmetic according to claim 10 wherein the cosmetic is emulsion type foundation.

13. Cosmetic according to claim 11 wherein the cosmetic is emulsion type foundation.

14. Cosmetic according to claim 12 wherein the emulsion type foundation is in the form of milky emulsion.

15. Cosmetic according to claim 13 wherein the emulsion type foundation is in the form of milky emulsion.

16. Cosmetic according to claim 12 wherein the emulsion type foundation is in the form of cream.

17. Cosmetic according to claim 13 wherein the emulsion type foundation is in the form of cream.

18. Cosmetic according to claim 1 which contains powdery pigments covered on the surface with the oily cosmetic composition and surface active agents.

19. Cosmetic according to claim 18 which is in the form of cake type foundation.

20. A process for producing cosmetic which comprises a process to produce oily cosmetic composition wherein silver type anti-bacterial agent is dispersed.

21. A process for producing cosmetic according to claim 20 wherein the silver type anti-bacterial agent is silver type anti-microbial water soluble glass.

22. A process for producing cosmetic according to claim 20 to produce oily foundation by mixing powdery pigments in dispersion in the oily foundation composition, and by forming into ointment or stick.

23. A process for producing cosmetic according to claim 20 to produce milky or creamy emulsion foundation by mixing in dispersion powdery pigments in either or both of the oily cosmetic composition and aqueous cosmetic composition, and by emulsifying the mixture with surface active agents.

24. A process for producing cosmetic according to claim 20 to produce milky or creamy emulsion foundation by making emulsion from the oily cosmetic composition and aqueous cosmetic composition with surface active agents, and then by mixing powdery pigments in dispersion in the emulsion.

25. A process for producing cosmetic according to claim 20 to produce cake type foundation by coating the surface of the powdery pigments with the oily cosmetic composition and surface active agents, and by arranging the particle size of the powders and by pressing them into solid cake.
